(19) United States
(12) Patent Application Publication
(10) Pub. No.: US 2019/0017079 A1
(43) Pub. Date: Jan. 17, 2019

(54) METHOD FOR FERMENTING SUGARS

(71) Applicant: CARGILL, INCORPORATED, Wayzata, MN (US)

(72) Inventors: Ignace André DEBONNE, Vollezele (BE); Ruben JOLIE, Herent (BE); Jean-Claude Marie Pierre Ghistlai DE TROOSTEMBERGH, Tielt-Winge (BE)

(73) Assignee: CARGILL, INCORPORATED, Wayzata, MN (US)

(21) Appl. No.: 16/067,215

(22) PCT Filed: Jan. 4, 2017
(86) PCT No.: PCT/US2017/012100

§ 371 Date: Jun. 29, 2018

(30) Foreign Application Priority Data
Jan. 5, 2016 (EP) 16150221.6
Aug. 3, 2016 (EP) 16182485.9

(51) Int. Cl. C12P 7/06; C12P 7/18; C12P 7/48

(52) U.S. Cl. CPC C12P 7/065; C12P 7/18; C12Y 204/01024; C12Y 302/01003; C12P 7/48

(57) ABSTRACT

In a first aspect of the present invention, there is provided a fermentation process for fermenting a carbon source comprising glucose and one or more oligosaccharides in the presence of a microorganism capable of fermenting glucose into a fermentation product, said process comprising the steps of: (a) forming an initial fermentation broth comprising the carbon source and the microorganism; (b) fermenting the broth under conditions suitable to ferment the glucose; (c) adding to the broth an effective amount of at least one active enzyme capable of depolymerizing the one or more oligosaccharides; and (d) recovering the fermentation product; wherein the carbon source comprises dextrose greens.

The work leading to this invention has received funding from the European Union Seventh Framework Programme (FP7/2007-2013) under grant agreement no FP7-KBBE-2013-7-613941.

### CROSS-REFERENCE TO RELATED APPLICATIONS

This application claims the benefit of European Patent Application No. 16150221.6, filed on Jan. 5, 2016, and European Patent Application No. 16182485.9, filed Aug. 3, 2016, both of which are incorporated by reference herein in their entirety.

### FIELD OF THE INVENTION

The present invention relates to a method for fermenting sugars. More particularly, the present invention relates to a method for fermenting sugars using dextrose greens as a carbon source.

### BACKGROUND OF THE INVENTION

Starch is readily available from a wide variety of plant sources such as corn, wheat, rice, potatoes, and barley. It consists of a large number of glucose units joined by glycosidic bonds and can therefore be hydrolysed to produce compositions rich in glucose (sometimes also known as “dextrose”). Such compositions are usually obtained through an enzymatic process involving liquefaction and saccharification (although they may also be obtained through acid conversion or a combination of the two).

During liquefaction, the starch molecules are gelatinized and converted to polysaccharides and oligosaccharides (also known as oligomeric polysaccharides or glucose oligomers) by the addition of a thermostable alpha-amylase enzyme to a starch slurry (for example, at about 35% dry solids, 100° C. and a pH of 5.8). The saccharides are then converted, during saccharification, to glucose by the addition (e.g., at about 60° C. and a pH of 4.5) of a glucoamylase enzyme together, optionally, with a starch debranching enzyme (e.g., pullulanase). The compositions obtained through this process are called “starch hydrolysates”. They may be further refined through vacuum filtration, ion exchange demineralization, and discoloration to remove impurities such as salts, proteins, lipids, organic acids, and fibers. They may also be concentrated to a dry solids content of e.g., about 70% by weight.

Starch hydrolysates typically contain 95% glucose by weight or more (based on total dry weight). They also contain some residual oligosaccharides. They may be used as a carbohydrate source for fermentation (see for example WO2005/100583) and are commonly used as a raw material in the production of crystalline dextrose. D-Glucose in the monohydrate form is produced by slow cooling of the starch hydrolysate (from 50 to 30° C., over two to three days). Anhydrous D-glucose is produced by evaporation crystallization (at 65° C.) under vacuum. Regardless of the method used, removal or recovery of glucose leaves behind a liquid by-product. This by-product is called “dextrose greens” (or “mother liquor”) and is typically sold to the animal feed industry as low quality, low cost by-products. They are, however, still relatively rich in carbohydrates (both glucose and oligosaccharides).

Many attempts have been made to recover the value of these residual carbohydrates. WO2014/047418, for example, describes a process comprising membrane filtration and enzymatic treatment of a glucose containing solution (such as a starch hydrolysate or mother liquor) to increase the yield of glucose production. WO2010/086840 describes a process to enhance ethanol yield from fermentation of molasses by the addition of depolymerising enzymes and xylose isomerase to the fermentation medium.

It is an object of the present invention to provide an alternative use for dextrose greens which would allow the full value of their carbohydrate content to be realized. This is achieved through fermentation.

Fermentation processes are used commercially at large scale to produce organic molecules such as ethanol, citric acid and lactic acid. In those processes, a carbohydrate is fed to an organism that is capable of using it to produce the desired fermentation product. The carbohydrate and organism are selected together so that the organism is capable of efficiently fermenting the carbohydrate to form the product that is desired in good yield. Glucose is an ideal carbohydrate source as it is easily and efficiently fermented by most microorganisms. Other oligo- and polysaccharides may also be fermented, but often only very slowly and inefficiently.

Because of their relatively high glucose content, starch hydrolysates have been considered a good source of glucose for fermentation. Conversely, because of their reduced glucose content and much higher oligosaccharide content (10-30% by weight based on total dry weight), dextrose greens have not been considered a good source. In fact, the presence of oligosaccharides in the fermentation medium is known to reduce fermentation yields and to complicate the recovery and purification of fermentation products. It is therefore considered prohibitive.

It is an object of the present invention to overcome these obstacles, to make full use of the carbohydrate content of dextrose greens through fermentation, and to improve downstream processing.

### STATEMENTS OF THE INVENTION

In a first aspect of the present invention, there is provided a fermentation process for fermenting a carbon source comprising dextrose greens, the dextrose greens comprising glucose and one or more oligosaccharides, in the presence of a microorganism capable of fermenting glucose into a fermentation product, said process comprising the steps of: (a) forming an initial fermentation broth comprising the carbon source and the microorganism; (b) fermenting the fermentation broth under conditions suitable to ferment the glucose; (c) adding to the broth an effective amount of at least one active enzyme capable of depolymerizing the one or more oligosaccharides; and (d) recovering the fermentation product.

### BRIEF DESCRIPTION OF THE FIGURES

FIG. 1 is a graph showing the residual level, in g/L, of different oligosaccharides (DP2, DP3 and DP4+) after S. cerevisiae fermentation according to Example 1.

FIG. 2 is a graph showing the residual level, in g, of different oligosaccharides (DP2, DP3 and DP4+) after E. coli fermentation according to Example 2.

### DETAILED DESCRIPTION OF THE INVENTION

The present invention relates to a fermentation process for fermenting a carbon source comprising glucose and one or more oligosaccharides, wherein the term “oligosaccharides” refers to oligomers of monosaccharides, linked by ether linkages (e.g. oligomers of glucose and/or fructose). The degree of polymerisation will typically be about 2 to 10 (referred to herein as DP2 to DP10 oligosaccharides).

### Carbon Source

The carbon source used in the process of the present invention comprises dextrose greens. As described above, dextrose greens (also known as “mother liquor”) are what remains after glucose has been recovered from a starch hydrolysate using any method known in the art (e.g., through crystallization or chromatography). The starch hydrolysate itself may be produced from any suitable starch source (such as corn, wheat, rice, barley, potatoes, cassava, and the like) by methods known in the art (typically including liquefaction and saccharification as described above).

Dextrose greens typically have a carbohydrate content of at least 95% by weight, based on total dry content. Preferably, they will have a carbohydrate content of at least 97%, more preferably at least 98%, more preferably at least 99% by weight. They will normally have a glucose content of 50-90% by weight, based on total carbohydrate (i.e., based on the total dry weight of carbohydrates in the composition). Preferably, they will have a glucose content of 60-85%, more preferably of 70-85%, for example a content of 75-85%, and, in some instances, a content of 80-85% by weight based on total carbohydrate.

In addition to glucose, they will also comprise 10-50% by weight oligosaccharides, based on total carbohydrate. Preferably, they will comprise 15-40%, more preferably 15-30%, more preferably 15-25% by weight oligosaccharides. The oligosaccharides will largely be disaccharides and trisaccharides. Preferably, they will be selected from the group consisting of: isomaltose, maltose, maltulose, panose, and mixtures of two or more thereof. More preferably, they will comprise each of these four oligosaccharides. Of course, other oligosaccharides may be present, but usually only in very small or trace amounts.

Preferably, each of isomaltose, maltose, and panose will be present in the dextrose greens in an amount of at least 2% by weight, based on total carbohydrate. More preferably, isomaltose and maltose will each be present in an amount of 2-10%, more preferably 2-7%, more preferably 2-5%, more preferably 3-5% by weight, and panose will preferably be present in an amount of 2-8%, more preferably 3-7%, more preferably 3-5% by weight, based on total carbohydrate. Maltulose will preferably be present in an amount of at least 0.5%, more preferably 0.5-10%, more preferably 1-7%, more preferably 2-5% by weight, based on total carbohydrate, and DP4+ saccharides will preferably be present in an amount of 0-5%, preferably 0.5-4%, more preferably 1-3% by weight, based on total carbohydrate.

Although the carbon source may comprise other carbohydrates (i.e., additional glucose and/or oligosaccharides from a source other than dextrose greens—including, for instance, residual sugars from the microbial inoculum), it will preferably comprise at least 90% dextrose greens by weight, more preferably 95%, more preferably 97%, more preferably 98%, more preferably 99%. Ideally, it will substantially consist of dextrose greens. In any event, the carbon source will preferably comprise 50-90% glucose by weight, and 10-50% oligosaccharides by weight, based on total carbohydrate—with preferred concentrations being as specified above for dextrose greens.

### Fermentation Broth

The carbon source is used to form a fermentation broth and, optionally, to supplement it during fermentation (as described below). The optimum quantity of glucose included in the fermentation broth will depend on the type of microorganism and the type of enzyme(s) being used, and will be readily determined by a person skilled in the art. By way of example, if the microorganism being used is E. coli, the glucose concentration may be about 30-40 g/L; for Saccharomyces, concentrations of 200 g/L or more may be possible.

In addition to the carbon source, the fermentation broth will typically also include water, a nitrogen source (such as proteins, ammonium sulphate, ammonia, urea or other nitrogen sources well known in the art) and other vitamins, salts and minerals. It may also comprise other components such as buffering agents and, as the fermentation progresses, fermentation products and certain metabolites.

The exact content of the broth will be adapted by a person skilled in the art, using common general knowledge, to ensure optimal growth of the microorganism being used, throughout the fermentation process. Thus, the broth will also comprise a microorganism capable of fermenting glucose into a fermentation product.

### Microorganism

The microorganism will be selected in relation to the desired fermentation product. It may be naturally occurring (so-called wild-type), or it may be a mutant or recombinant strain. Examples of suitable microorganisms include various species of fungi (such as Saccharomyces, Aspergillus, Kluyveromyces, Penicillium, Pichia, Hansenula, Candida, Trichosporon, Issatchenkia, Yamadazyma, Rhizopus, Yarrowia, Moniliella), bacteria (such as Lactobacillus, Lactococcus, Streptococcus, Pediococcus, Staphylococcus, Leuconostoc, Streptomyces, Bacillus, Paenibacillus, Escherichia, Clostridium, Xanthomonas, Pseudomonas, Acetobacter, Gluconobacter, Zymomonas, Klebsiella, Enterobacter, Thermotoga, Brevibacterium, Ketogulonicigenium), algae, and archaea. Preferably, the microorganism will be selected from Saccharomyces cerevisiae (S. cerevisiae), Issatchenkia orientalis (also known as Pichia kudriavzevii), and Escherichia coli (E. coli).

Because microorganisms are typically unable to metabolize oligosaccharides present in dextrose greens, at least one active enzyme capable of depolymerizing the one or more oligosaccharides will be added to the fermentation broth.

### Enzyme

The enzyme may be any enzyme effective for depolymerizing oligosaccharides having a 1→4 and/or a 1→6 ether linkage. Suitable enzymes include glucoamylase (EC 3.2.1.3), transglucosidase (EC 2.4.1.24), isomaltase (EC 3.2.1.10), alpha-glucosidase (EC 3.2.1.20), pullulanase (EC 3.2.1.41), isoamylase (EC 3.2.1.68) and mixtures of two or more thereof. Preferred enzymes include glucoamylase and transglucosidase, or, even more preferably, a combination of both. It was indeed surprisingly found that while glucoamylase is very effective for depolymerizing the most common DP2 oligosaccharides present in starch hydrolysates (such as maltose) and is very effective for depolymerizing DP3 and DP4+ oligosaccharides characteristic of dextrose greens, it only has a limited effect on the DP2 oligosaccharides characteristic of dextrose greens, specifically isomaltose (2 glucoses in 1→6 bond) and maltulose (a glucose and a fructose in a 1→4 bond). By contrast, it has been found that transglucosidase is very effective for depolymerizing the DP2 and DP3 oligosaccharides present in dextrose greens (such as maltulose), with only a limited effect on its DP4+ oligosaccharides. As such, glucoamylase and transglucosidase will advantageously be used together to optimise the carbohydrates available for fermentation from dextrose greens. The enzymes may be added simultaneously or sequentially. For example, glucoamylase may be added to the fermentation broth first, followed by transglucosidase (e.g., after glucose levels have been reduced through fermentation). For clarity, it should be noted that the terms “transglucosidase” and “alpha-glucosidase” are sometimes used interchangeably in the art as transglucosidase enzymes may exhibit alpha-glucosidase activity under certain conditions.

The amount of enzyme to be used depends on the selected enzyme(s), the selected enzyme preparation, the desired rate of reaction, and the reaction conditions, including the concentration and type of oligosaccharides present in the fermentation broth. Typically, the enzyme is used in a quantity sufficient to provide about 5-10,000 μL of liquid enzyme preparation/L of fermentation broth (it being understood that liquid enzyme preparations typically comprise between 5 and 20% enzyme by weight). A more preferred quantity is from about 10-1000 μL/L and an even more preferred quantity is from about 25-500 μL/L (i.e. an amount of enzyme of about 1.5 to 115 mg per kg broth).

Glucoamylase will preferably be used in an amount of 25-1500 μL/L, more preferably in an amount of 50-1000 μL/L, more preferably in an amount of 75-750 μL/L, more preferably in an amount of 100-500 μL/L, more preferably in an amount of 150-250 μL/L of fermentation broth.

Advantageously, transglucosidase will be used in an amount of more than 25 μL/L, preferably 50 μL/L or more, more preferably 75 μL/L or more, more preferably 100 μL/L or more. For example, transglucosidase may be used in an amount of 100-1500 μL/L, preferably in an amount of 150-1000 μL/L, more preferably in an amount of 200-750 μL/L, more preferably in an amount of 250-500 μL/L of fermentation broth.

When used together, glucoamylase and transglucosidase will advantageously be present in a weight ratio of 2:1 to 1:2, more preferably of 3:2 to 2:3, more preferably of approximately 1:1. As noted above, glucoamylase and transglucosidase may be used simultaneously or sequentially. If used sequentially, glucoamylase will preferably be added to the fermentation broth before transglucosidase. Indeed, while transglucosidase may be sensitive to high concentrations of glucose, this is not equally true for glucoamylase. As such, by adding glucoamylase first, there will be no need to limit the concentration of glucose in the fermentation broth. Transglucosidase can then be added only when the glucose concentration reaches, for example, 30 g/L or less.

These concentrations are used to provide a good approximation of the amount of enzyme to be added to the fermentation broth, which is evaluated by experimental observation to determine actual amounts of enzyme required under conditions of use (e.g. temperature, pH and other conditions particular to a given fermentation process) to achieve the desired concentrations of residual oligosaccharides in the final fermentation broth at the lowest cost of enzymes.

### Fermentation Product

The process of the present invention may be used to produce any product that can be obtained through fermentation. It will be particularly beneficial for the preparation of fermentation products for use in non-food applications. Although it may be unexpected, some residual ingredients of the fermentation process that may be present in foods (e.g., residual oligosaccharides) may be problematic for non-food applications. In particular, they may complicate the separation, recovery and purification of fermentation products by causing undesirable reactions, either with other components of the fermentation medium (which, in turn, can lead to the formation of difficult to remove impurities), or with the fermentation products themselves (leading to losses in recovery yield).

Examples of possible fermentation products include amino acids, organic acids, alcohols, diols, polyols, fatty acids, monoacylglycerols, diacylglycerols, triacylglycerols, polysaccharides (such as xanthan, scleroglucan and schizzophylan), microbial biomass and mixtures thereof. Preferred fermentation products include organic acids, diols, amino acids and salts thereof. Examples of organic acids that may be produced according to the present invention include hydroxyl carboxylic acids, hydroxyl polycarboxylic acids, dicarboxylic acids, tricarboxylic acids and mixtures thereof. Preferred organic acids include lactic acid, citric acid, malonic acid, hydroxy butyric acid, adipic acid, keto-glutaric acid, glutaric acid, 3-hydroxy-propionic acid, succinic acid, malic acid, fumaric acid, itaconic acid, muconic acid, methacrylic acid, and acetic acid, together with derivatives and salts thereof. Other preferred fermentation products include, for instance, ethanol, propanediol (PDO) and butanediol (BDO). Others possible products will be apparent to a person skilled in the art.

### Process

As noted above, the present invention provides a fermentation process comprising the following steps:
(a) forming an initial fermentation broth comprising a carbon source and a microorganism, wherein the carbon source comprises dextrose greens;
(b) fermenting the broth under conditions suitable to ferment the glucose;
(c) adding to the broth an effective amount of at least one active enzyme capable of depolymerizing the one or more oligosaccharides; and
(d) recovering the fermentation product,
   wherein each of fermentation broth, carbon source, microorganism, dextrose greens, enzyme, oligosaccharides, and fermentation product are all as defined above; and wherein step (c) will preferably be performed during step (b) or wherein step (b) will preferably be continued after step (c).

This process will be performed under conditions that allow fermentation to occur. These conditions will be well understood by the skilled person and may vary depending on the particular organism being used and the desired fermentation product. For reference, typical conditions include a temperature of above 20° C., preferably of above 30° C., more preferably of about 25° C. to about 50° C., more preferably of about 30° C. to about 40° C. (e.g. about 35° C.). In addition, the fermentation broth will usually be mixed (e.g. by sparging gas into the broth or, alternatively, by direct mechanical agitation or other means). The fermentation will typically be performed in a bioreactor which allows these conditions to be easily monitored and controlled.

Preferably, step (b) will be continued until depletion of substantially all the fermentable sugars (both glucose and oligosaccharides) from the fermentation broth. Ideally, this means that fermentation will be continued until no further metabolic activity is observed or until the glucose concentration in the fermentation broth reaches less than 5 g/L, preferably less than 3 g/L, more preferably less than 2 g/L, more preferably less than 1 g/L, more preferably no more than about 0.5 g/L, more preferably about 0 g/L. For the sake of clarity, this means that step (b) may continue simultaneously with (and subsequently to) step (c).

While the process of the invention may be a batch process (in which nothing is added to the fermentation broth after fermentation has been initiated and product is recovered only at completion), it may also be a fed-batch process (in which nutrients are added in increments as the fermentation progresses), or a continuous process (in which nutrients are added to, and product is removed from, the fermentation broth in a continuous manner during fermentation). Further details of such processes are provided below. Preferably, the process will be a batch or a batch-fed process.

For instance, it may be possible to supplement the fermentation broth with additional fermentable sugars. Thus, the process of the present invention may include a further step of adding further glucose to the fermentation broth (either before, simultaneously with, or after starting enzyme addition). The additional glucose will preferably be delivered in the form of additional dextrose greens, but may also be in the form of a starch hydrolysate or glucose syrup. It may be added in a single step (“one shot” addition), or progressively over a certain period of time (e.g., in increments). The rate of addition will be determined by the skilled person to ensure that it is balanced with the speed of use by the microorganism. By way of example only, it may be added at a rate of 1-10 g glucose per hour. Advantageously, additional glucose will be added to the fermentation broth to maintain a desired glucose concentration in the fermentation broth. This may be, for example, from about 1 to about 10 g/L, more preferably of about 1 to about 5 g/L.

Whether additional substrate is introduced into the fermentation broth or not, the process of the invention may continue, for example, until a desired quantity of fermentation product has been produced or until the microorganisms are no longer effective (high concentrations of fermentation product can have an inhibitory effect on the microorganisms). Alternatively, as additional substrate is added, fermentation broth (including fermentation product) may be bled from the bioreactor, allowing the fermentation process to become continuous.

Enzyme will preferably be added under conditions that permit the simultaneous fermentation of glucose and depolymerization of oligosaccharides. It may be added in a single addition step or progressively over a certain period of time. For example, its addition may be metered over time from about 3 minutes to about 3 hours. It may also be added in a plurality of addition steps over the course of the full fermentation process. For example, the broth may be monitored to measure oligosaccharide concentrations and additional enzyme added as needed to achieve the desired depolymerization levels.

Under certain conditions, it has been observed that the addition of depolymerizing enzyme to highly concentrated glucose solutions (e.g., a fermentation broth with a glucose concentration above 30 g/L) can render the enzyme less effective and perhaps even trigger reversion or condensation reactions (e.g., the formation rather than the depolymerization of oligosaccharides). This may be the case, for example, for transglucosidase. It is also known that many enzymes have finite times of effectiveness and so later addition of an enzyme to the fermentation broth may be advantageous to make optimal use of its active lifetime. It may therefore be beneficial to start the fermentation in the absence of enzyme (or in the absence of enzyme sensitive to high glucose concentrations) and to only add it once a more favourable glucose concentration has been reached. Such a glucose concentration will advantageously be below 30 g/L, preferably below 25 g/L, more preferably below 20 g/L, more preferably below 15 g/L. However, it is also possible to add some, or all, of the enzyme before starting the fermentation process, i.e. simultaneously with or immediately after addition of the carbon source. Thus, step (c) above may also be performed before the beginning of step (b) and, for clarity, the present invention provides a process whereby:
a) an initial fermentation broth comprising a carbon source and a microorganism is formed, wherein the carbon source comprises dextrose greens;
b) an effective amount of at least one active enzyme capable of depolymerizing the one or more oligosaccharides present in the carbon source is added;
c) the broth is fermented under conditions suitable to ferment the glucose and to depolymerize the oligosaccharides from the carbon source; and
d) the fermentation product is recovered.

The present invention also provides a process whereby:
a) an initial fermentation broth comprising a carbon source and a microorganism is formed, wherein the carbon source comprises dextrose greens;
b) an effective amount of a first enzyme (such as glucoamylase) is added;
c) the broth is fermented until a glucose concentration below 30 g/L is achieved;
d) an effective amount of a second enzyme (such as transglucosidase) is added;
e) fermentation is continued; and
f) the fermentation product is recovered.

How the fermentation product is recovered will depend on the nature of the fermentation product to be recovered. In general, the microorganism will be separated from the fermentation broth, typically via a filtration or centrifugation step, and the fermentation product will then be recovered via, for example, distillation, extraction, crystallization, membrane separation, osmosis, reverse osmosis, evaporation, or other suitable means well known to the person skilled in the art. The fermentation product may be recovered at the end of the fermentation process or during the fermentation process itself (e.g., in a continuous process).

Advantageously, the process of the present invention allows improved yields of fermentation product to be achieved from dextrose greens as more of the carbon source is converted to fermentable sugars. It also facilitates recovery of the fermentation product as the significantly reduced oligosaccharide content will facilitate separation and purification, and cause fewer undesirable reactions with the fermentation product and other components of the fermentation medium (thereby ensuring an optimum yield and fewer impurities) during the recovery process.

The above aspects of the present invention are not intended to be exhaustive or to limit the invention to the precise forms disclosed. Rather, a purpose of this description is so that an appreciation and understanding by others skilled in the art of the principles and practices of the present invention can be facilitated. The present invention will now be further described in the following, non-limiting examples.

### EXAMPLES

### Carbon Source

Samples of both high glucose starch hydrolysate and dextrose greens were analyzed (using HPLC-RID with dual Shodex KC-811 (H⁺ form)) and their approximate carbohydrate composition is set out below (in percent by weight based on total carbohydrate):

| | % Dry | | | | |
|---|---|---|---|---|---|
| Sample | Substance | % DP1 | % DP2 | % DP3 | % DP4+ |
| C*Sweet D02761 | 70.8 | 96.0 | 2.5 | 1.0 | 0.5 |
| (high glucose | | | | | |
| starch hydrolysate | | | | | |
| from Cargill) | | | | | |
| C*Sweet D15080 | 70.7 | 84.8 | 9.3 | 4.1 | 1.8 |
| (dextrose greens | | | | | |
| from Cargill) | | | | | |
| batch 1 | | | | | |
| C*Sweet D15080 | 71.9 | 82.3 | 11.0 | 4.9 | 1.8 |
| (dextrose greens | | | | | |
| from Cargill) | | | | | |
| batch 2 | | | | | |

For both the high glucose starch hydrolysate and the dextrose greens, the DP1 products were almost exclusively glucose. The DP2 products were mostly maltose, isomaltose and maltulose (ca. 0.5-1.0% of each for the high glucose starch hydrolysate and ca. 2.0-3.5% of each for the dextrose greens). The DP3 products were mostly panose for both the high glucose starch hydrolysate (ca. 0.8%) and the dextrose greens (ca. 3.5-4%).

### Enzymes

The following enzymes were tested:

| Name | Class | Preparation | Main Action* |
|---|---|---|---|
| Glucoamylase | E.C. | Distillase ® CS | Hydrolysis of terminal |
| (GA) | 3.2.1.3 | (DuPont) | (1−>4)-linked α- |
| | | | D-glucose residues |
| | | | successively from |
| | | | non-reducing ends |
| | | | of the dextrin or |
| | | | oligosaccharide chain |
| | | | with release of β- |
| | | | D-glucose |
| Transglucosidase | E.C. | GC444 or | Transfer of an α- |
| (TG) | 2.4.1.24 | Fermenzyme ® T | D-glucosyl residue in |
| | | (DuPont) | a (1−>4)-α-D- |
| | | [previously | glucan to the primary |
| | | referred to as | hydroxyl group of |
| | | Transglucosidase L | glucose, free or |
| | | 2000] | combined in a (1−>4)- |
| | | | α-D-glucan |

*see http://www.brenda-enzymes.org/index.php

### Microorganisms

Two microorganisms were used in the tests:
1) Saccharomyces cerevisiae, Thermosacc® Dry (Lallemand), to convert glucose into biomass, ethanol and CO₂ via batch fermentation
2) an E. coli B-strain, to convert glucose into biomass and CO₂ via fed-batch fermentation

### Example 1

### Saccharomyces cerevisiae Fermentation

Shake flasks were used at 32° C. with a reference working volume of 0.2 L.

### Batch Process

Carbon source (C*Sweet D02761 or C*Sweet D15080 as described above): circa 200 g carbohydrate/kg, all added to initial fermentation broth
Nitrogen source: yeast extract and urea
Yeast inoculum
pH at start: 4.5 (not controlled during batch process)
Addition of enzyme(s) after 24 and/or 48 h (when glucose reaches less than 30 g/L)
Fermentation continued until 72 h (complete depletion of fermentable sugars)

To assess the effect of the enzymes, fermentations were run without enzyme, with only GA or TG, and with both GA and TG, as follows:

| | No | | | GA at 24 h + |
|---|---|---|---|---|
| | Enzyme | TG at 24 h | GA at 24 h | TG at 48 h |
| C*Sweet D02761 | ✓ | | | |
| C*Sweet D15080 | ✓ | ✓ | ✓ | ✓ |

GA was used at 100 μL per kg broth or 0.56 g per kg carbohydrate, and TG was used at 150 μL per kg broth or 0.85 g per kg carbohydrate.

### Measurements

Sugar composition was measured at completion of the fermentation process, with each fermentation being run for the same amount of time. Glucose, fructose, DP2, DP3 and DP4+ (DP2, DP3 and DP4+ referred to, together, as DP2+) were measured by HPLC-RID with dual Shodex KC-811 (H⁺ form) column and H2SO4 eluent. Maltose, isomaltose, maltulose and panose were measured by HPAEC-PAD with CarboPac PA-20 column and NaOH eluent. Enzyme and bacterial activity were quenched immediately after sampling by subjecting samples to a heat shock;
Ethanol titers were measured at end of fermentation (HPLC-RID with dual Shodex KC-811 (H⁺ form) column and H2SO4 eluent);
Ethanol yield on total carbohydrate was calculated according to the following formula: 100×final ethanol/total carbohydrate;
Estimated percent carbohydrate conversion (or “fermentability”) was calculated according to the following formula: 100×(total carbohydrate−residual DP2+)/total carbohydrate.

### Results

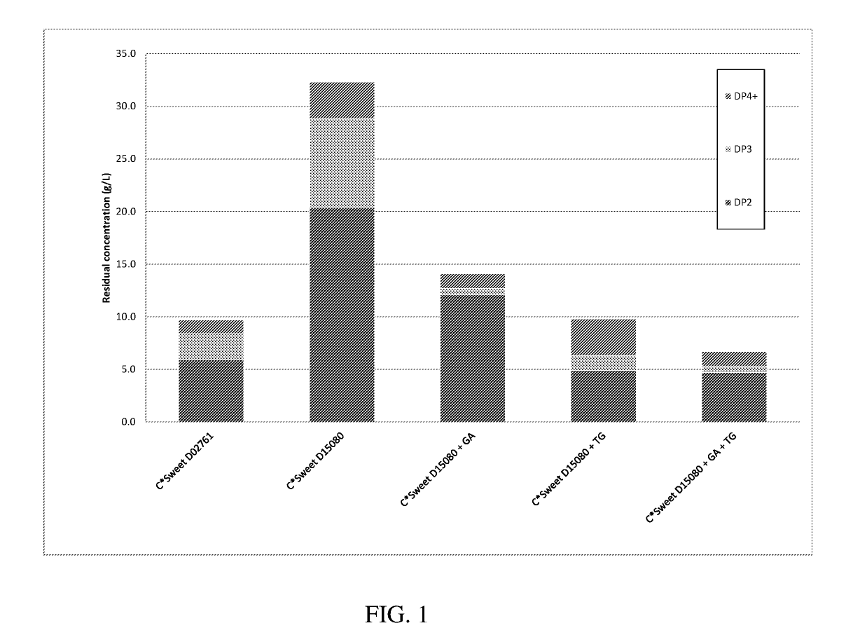

Results of the above measurements are shown in the tables below, and in FIG. 1 (results are shown as mean results for repeated trials on different feedstock batches):

| Based on | DP2 | DP3 | DP4+ | DP2+ | Fermentability | Ethanol |
|---|---|---|---|---|---|---|
| HPLC-RID | (g/L) | (g/L) | (g/L) | (g/L) | (%) | yield |
| C*Sweet D02761 | 5.9 | 2.5 | 1.3 | 9.7 | 95.3 | 48.1 |
| C*Sweet D15080 | | | | | | |
| w/o enzyme | 20.4 | 8.4 | 3.5 | 32.3 | 84.2 | 42.7 |
| +GA_{at 24 h} | 12.1 | 0.6 | 1.4 | 14.1 | 93.1 | 47.7 |
| +TG_{at 24 h} | 4.9 | 1.4 | 3.5 | 9.8 | 95.2 | 47.5 |
| +GA_{at 24 h} | 4.7 | 0.6 | 1.4 | 6.7 | 96.7 | 48.7 |
| and TG_{at 48 h} | | | | | | |

| | Maltose | Isomaltose | Maltulose | Panose |
|---|---|---|---|---|
| Based on HPAEC-PAD | (g/L) | (g/L) | (g/L) | (g/L) |
| C*Sweet D02761 | 1.6 | 2.2 | 1.6 | 2.0 |

C*Sweet D15080

| | | | | |
|---|---|---|---|---|
| w/o enzyme | 6.2 | 6.7 | 3.8 | 7.1 |
| +GA_{at 24 h} | <0.1 | 4.9 | 3.7 | <0.1 |
| +TG_{at 24 h} | <0.1 | 0.3 | 2.5 | 0.2 |
| +GA_{at 24 h} and TG_{at 48 h} | <0.1 | 0.2 | 2.5 | <0.1 |

The results show that, without the addition of enzyme, the fermentability of and ethanol yield from dextrose greens is far lower than for high glucose starch hydrolysate. The addition of GA, or TG, however, allows a comparable fermentability and yield to be obtained. More surprisingly, the addition of both GA and TG to the same fermentor was found to show, not just comparable, but better fermentability and yield. This improvement is associated with reduced levels of residual oligosaccharides (DP2+). GA addition enables strong reduction in residual maltose, panose and DP4+ concentrations, while TG addition lowers residual maltose, isomaltose, maltulose and panose levels. Adding both enzymes allows reducing maltose, isomaltose, maltulose, panose and DP4+ levels in the final fermentation broth.

### Example 2

### E. coli Fermentation

Fermenters were set up at 37° C. with a reference working volume of 1.3 L.

### Fed-Batch Process

Phase 1:
Carbon source (C*Sweet D02761 or C*Sweet D15080 as described above, diluted in water)
Nitrogen source: ammonium sulfate and ammonia (also for pH control at 6.0)
Salts, minerals
Bacterial inoculum
Phase 2:
Linear sterile addition of extra carbon source (high glucose starch hydrolysate or dextrose greens as described above, diluted in water) during 48 h, starting after depletion of initial carbon source from batch
Single shot addition of enzyme, added simultaneously with the start of carbon source addition for Phase 2
Continued until complete depletion of fermentable sugars (no more metabolic activity)

| | | Carbon | | | |
|---|---|---|---|---|---|
| | Volume | Source | Carbohydrates | Carbohydrates | Glucose |
| | (L) | (g) | (g) | (g/L) | (g/L) |
| Phase 1 | 0.863 | 43.9 | 31.6 | 36.6 | 30.2^{a} or |
| | | | | | 35.3^{b} |
| Phase 2 | 0.437 | 317.7 | 228.7 | | |
| Total | 1.300 | 361.5 | 260.3 | 200.2 | 165^{a} or |
| | | | | | 193^{b} |

^{a}for C*Sweet D15080 and
^{b}for C*Sweet D02761

To assess the effect of the enzymes when fermenting on dextrose greens, fermentations were run without enzyme, with only GA or TG, and with both GA and TG, as follows (where “low”=0.25 mL/L final broth or 1.0 g per kg syrup, and “high”=1 mL/L final broth or 4.1 g per kg syrup):

| | No Enzyme | TG | GA | TG + GA |
|---|---|---|---|---|
| C*Sweet D02761 | ✓ | | | |
| C*Sweet D15080 | ✓ | ✓ low | ✓ low | ✓ both low |
| | | | ✓ high | ✓ both high |

### Measurements

Sugar composition was measured at completion of the fermentation process, with each fermentation being run for the same amount of time. Glucose, fructose, DP2, DP3 and DP4+ (DP2, DP3 and DP4+ referred to, together, as DP2+) were measured by HPLC-RID with dual Shodex KC-811 (H⁺ form) column and H2SO4 eluent. Maltose, isomaltose, maltulose and panose were measured by HPAEC-PAD with CarboPac PA-20 column and NaOH eluent. Enzyme and bacterial activity were quenched immediately after sampling by subjecting samples to a heat shock;
Estimated percent carbohydrate conversion (or “fermentability”) was calculated according to the following formula: 100×(total carbohydrate−residual DP2+)/total carbohydrate.

### Results

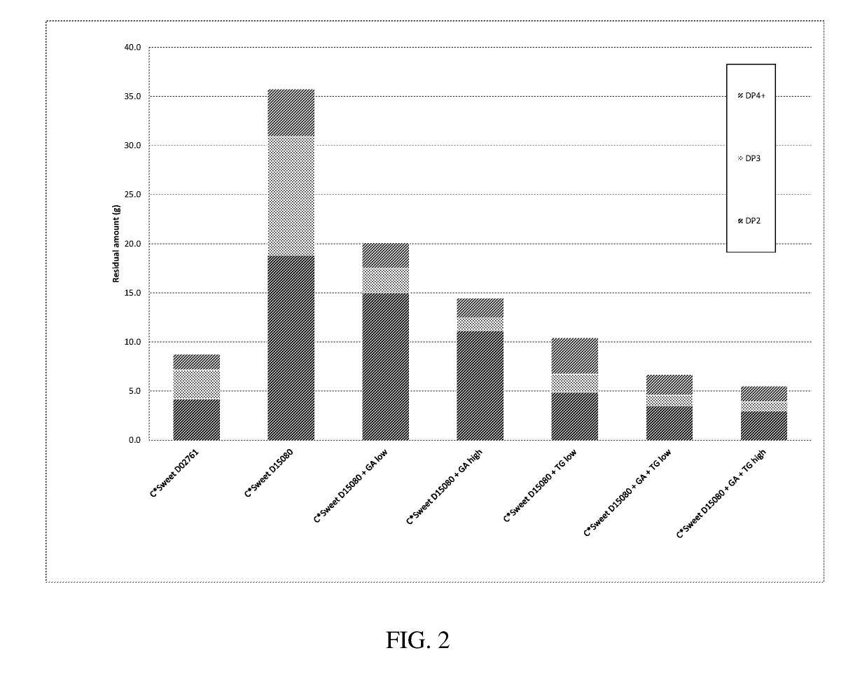

Results of the above measurements are shown in the tables below, and in FIG. 2 (results are shown as mean results for repeated trials on different feedstock batches):

| | DP2 | DP3 | DP4+ | DP2+ | Fermentability |
|---|---|---|---|---|---|
| Based on HPLC-RID | (g) | (g) | (g) | (g) | (%) |
| C*Sweet D02761 | 4.2 | 3.0 | 1.6 | 8.8 | 96.6 |

C*Sweet D15080

| | | | | | |
|---|---|---|---|---|---|
| w/o enzyme | 18.8 | 12.1 | 4.9 | 35.8 | 86.2 |
| +GA_{low} | 15.0 | 2.5 | 2.6 | 20.1 | 92.3 |
| +GA_{high} | 11.2 | 1.3 | 2.0 | 14.5 | 94.4 |
| +TG_{low} | 4.9 | 1.9 | 3.7 | 10.4 | 96.0 |
| +TG + GA_{low} | 3.5 | 1.1 | 2.1 | 6.7 | 97.4 |
| +TG + GA_{high} | 3.0 | 0.9 | 1.6 | 5.5 | 97.9 |

| | Maltose | Isomaltose | Maltulose | Panose |
|---|---|---|---|---|
| Based on HPAEC-PAD | (g) | (g) | (g) | (g) |
| C*Sweet D02761 | <0.1 | 2.4 | 1.4 | 1.9 |

C*Sweet D15080

| | | | | |
|---|---|---|---|---|
| w/o enzyme | <0.1 | 8.8 | 6.6 | 9.0 |
| +GA_{low} | <0.1 | 6.7 | 4.4 | 0.8 |
| +GA_{high} | <0.1 | 3.9 | 3.5 | <0.1 |
| +TG_{low} | <0.1 | 0.3 | 1.9 | 0.2 |
| +TG + GA_{low} | <0.1 | 0.1 | 0.9 | <0.1 |
| +TG + GA_{high} | <0.1 | <0.1 | <0.1 | <0.1 |

As can be seen from these results, compared to high glucose starch hydrolysate, when fermenting dextrose greens in the absence of enzyme, significantly more (oligo)saccharides remain at the end of the fermentation (i.e. resulting in wasted carbohydrate and possible complications to downstream processing). Adding enzyme reduced this waste. GA on its own resulted in a strong reduction in panose and DP4+ and a moderate reduction in isomaltose and maltulose. TG on its own resulted in a strong reduction in isomaltose, maltulose and panose and a moderate reduction in DP4+. Together, GA and TG brought a strong reduction in isomaltose, maltulose, panose and DP4+, even reaching levels below those obtained for high glucose starch hydrolysate. It was also observed that high enzyme doses resulted in stronger reductions in oligosaccharide levels.

The fermentability results confirm that the fermentability of dextrose greens increases when fermenting in the presence of enzymes, and that levels of fermentability above those obtained with high glucose starch hydrolysates can be obtained when using a combination of both TG and GA.

1. A fermentation process for fermenting a carbon source comprising glucose and one or more oligosaccharides in the presence of a microorganism capable of fermenting glucose into a fermentation product, said process comprising the steps of:
a) Forming a fermentation broth comprising the carbon source and the microorganism;
b) Fermenting the broth under conditions suitable to ferment glucose;
c) Adding to the broth an effective amount of at least one active enzyme capable of depolymerizing the one or more oligosaccharides; and
d) Recovering the fermentation product;
wherein the carbon source comprises dextrose greens.

2. A process according to claim 1, characterized in that the carbon source comprises 90% dextrose greens by weight.

3. A process according to claim 1, characterized in that the carbon source comprises 50-90% glucose by weight, based on total carbohydrate.

4. A process according to claim 1, characterized in that the carbon source comprises at least 10-50% by weight of the one or more oligosaccharides, based on total carbohydrate.

5. A process according to claim 1, characterized in that the one or more oligosaccharides comprise disaccharides and trisaccharides, preferably selected from the group consisting of: isomaltose, maltose, maltulose, panose, and mixtures of two or more thereof.

6. A process according to claim 1, characterized in that the oligosaccharides comprise isomaltose, maltose, maltulose and panose.

7. A process according to claim 1, characterized in that each of isomaltose, maltose, and panose are present in an amount of at least 2% by weight, based on total carbohydrate and maltulose is present in an amount of at least 0.5% by weight, based on total carbohydrate.

8. A process according to claim 1, characterized in that the microorganism is selected from strains of Escherichia coli and Saccharomyces cerevisiae.

9. A process according to claim 1, characterized in that the fermentation product is selected from ethanol, propanediol, butanediol, citric acid, lactic acid, and itaconic acid.

10. A process according to claim 1, characterized in that the enzyme is selected from glucoamylase, transglucosidase and mixtures thereof.

11. A process according to claim 10, characterised in that glucoamylase is used in an amount of 25-1500 μL/L fermentation broth.

12. A process according to claim 10, characterised in that transglucosidase is used in an amount of more than 25 μL/L fermentation broth.

13. A process according to claim 10, characterised in that glucoamylase and transglucosidase are used in a weight ratio of 2:1 to 1:2.

14. A process according to claim 1, characterized in that step (c) is performed before step (b) has been initiated.

15. A process according to claim 1, characterized in that step (c) is performed in increments, preferably until substantially all oligosaccharides have been depolymerized.

16. A process according to claim 1, characterized in that it comprises the addition of further carbon source during fermentation step (b).

17-18. (canceled)
